(19) United States
(12) Patent Application Publication
(10) Pub. No.: US 2021/0386431 A1
(43) Pub. Date: Dec. 16, 2021

(54) ENDOVASCULAR, RAPID-ACCESS, BALLOON CATHETER FOR TREATMENT OF PULMONARY EMBOLISM

(71) Applicant: Ashanga Yatawatta, Bullhead City, AZ (US)

(72) Inventor: Ashanga Yatawatta, Bullhead City, AZ (US)

(21) Appl. No.: 17/303,923

(22) Filed: Jun. 10, 2021

Related U.S. Application Data
(60) Provisional application No. 62/705,077, filed on Jun. 10, 2020.

(51) Int. Cl. A61B 17/12

(52) U.S. Cl. CPC A61B 17/12136; A61B 2017/00292; A61B 2217/005

(57) ABSTRACT

A transcatheter treatment device for pulmonary embolism and a method of using the same is provided. The treatment device has a conduit extending between an expandable balloon and an aspirator inlet, therebetween the conduit provides one or more outlets fluidly coupled to lytic agents. The expandable balloon is adapted to be actuated between a collapsed condition and an expanded condition dimensioned to seal off a portion of the interior of the pulmonary artery. In the collapsed condition the treatment device is percutaneously inserted into the pulmonary artery over a wire until the aspirator inlet is adjacent the pulmonary embolism, wherein the expandable balloon actuates, sealing off a pulmonary bay between the expanded balloon and the occluding blockage, as which time the practitioner can selectively deliver lytic agents and suction into this closed system space of the pulmonary artery in such a way as to disintegrate the clot.

### CROSS-REFERENCE TO RELATED APPLICATION

This application claims the benefit of priority of U.S. provisional application No. 62/705,077, filed 10 Jun. 2020, the contents of which are herein incorporated by reference.

### BACKGROUND OF THE INVENTION

The present invention relates to pulmonary embolism treatments and, more particularly, an endovascular, rapid-access, balloon catheter for treatment of pulmonary embolism.

Pulmonary embolism is common and fatal with minimal treatment options for effective, timely treatment, where time is of the essence. Pulmonary embolism is the leading cause of death in hospitals worldwide. Treatment is delayed and difficult due to a narrow therapeutic window and difficulty in gaining access in a timely manner.

Current approaches for treating pulmonary embolism are fraught with low efficacy and high complication rates, as well as being time consuming, resulting in a high mortality rate. The treatment options for pulmonary embolism include open chest surgery, and high dose lytic therapy, both of which has have low rates of success. The few endovascular options use mechanical disruption methods that require experts in specific locations which are not readily available.

Current treatments of pulmonary embolism have disadvantages. Open thoracotomy is invasive with significant morbidity and mortality and involves significant delays due to the resources needed. Systemic thrombolysis lacks accuracy with significant limitations and complications due to effects away from the intended site. Thrombectomy with the traditional endovascular approach is performed in a dedicated endovascular suite usually under anesthesia and needs resources that may not be available at bedside. In short, current treatments for pulmonary embolism are resource heavy, maximally invasive, require specialized training, and not readily available in all hospital at any given time.

As can be seen, there is a need for an endovascular, rapid-access, balloon catheter for treatment of pulmonary embolism. The present invention embodies Interventional radiology to deliver a minimally invasive treatment using medical imaging guidance, such as x-ray fluoroscopy, computed tomography, magnetic resonance imaging, or ultrasound. Accordingly, the method of the present invention is easily deployed at bedside.

The systemic device embodied by the present invention includes a catheter operatively associated with an expandable membrane, such as a balloon of the like, wherein the membrane can selectively be moved between a collapsed condition and an expanded condition for creating a closed system. The systemic device also provides an inlet at a distal end thereof, wherein the inlet provides suction. Just inward of the inlet may be one or more outlet adapted to selectively provide suitably high dose lytics.

As a result, the expanded expandable membrane is dimensioned and adapted to close off a section of a biological space into which the inlet may infuse high-dose lytics while the inlet aspirates the biological space. The one or more outlets may be two outlets disposed along opposing sides of an elongated portion of the system device so that the infusion follows an orbital path about the inlet/distal end of the systemic device. Systemic side-effects are minimal due to the closed system and the rapid clot disruption due to localized lytics. The orbital path is urged by the distal suction, much like a magnetic field is defined by a magnetic pole.

The new method may be a minimally invasive approach used to access the internal jugular vein in the neck under ultrasound guidance. The systemic device may be deployed via a catheter over a wire to a pulmonary bay, which may be the biological space from which the closed system is defined through expanding the expandable membrane, thereby isolating the pulmonary artery in the closed system of the biological space. The pulmonary bay may be a concavity or cavity adjacent to the pulmonary artery resulting from a reduced outflow from the pulmonary artery caused by a pulmonary embolism.

The closed system may then be subjected to high dose lytic infusion and suction at the same time. This localized environment allows lysis and breakup of the blot clots away from the pulmonary bay, and thereby reducing immediate mortality.

The new method improves on the access, simplicity, and effectiveness of existing methods with the use of high dose lytic and suctions in a closed system generated by a balloon occlusion. The present invention embodies a minimally invasive treatment that can be done at bedside on critically Ill patients with use of simple image guidance. Furthermore, this approach does minimal additional physiological insult with minimal anesthesia and can be tailored according to the level of the patient's sickness as well as thrombus load. Additionally, the method embodied in the present invention can be tailored according to location with response monitoring leading to real time adjustments.

The present invention can be used at bedside with local anesthesia. It allows introduction of a catheter with a balloon tip from the internal jugular vein and allows mechanical aspiration of clots with continuous thrombolytic chemicals introduced at a different opening of the catheter. Pharmaco-mechanical thrombectomy and thrombolysis can be performed either as an initial first stage, few stages over prolonged time periods, or as a continuous low intensity method depending on the patient and their thrombus characteristics. This method minimizes systemic release of lytic agents due to suction aspiration and can be guided by ultrasound, echo and fluoroscopy in real time at bedside—providing advantages over the prior art.

### SUMMARY OF THE INVENTION

In one aspect of the present invention, a device for endovascular treatment for a patient with a pulmonary embolism includes the following: a conduit extending between an expandable balloon and an aspirator inlet; the expandable balloon selectively movable between a collapsed condition and an expanded condition dimensioned to seal an arterial space defined by the pulmonary artery of the patient; and one or more outlets circumferentially disposed along the conduit between the expandable balloon and the aspirator inlet. In another aspect of the present invention, the endovascular treatment device includes wherein the aspirator inlet generates suction, wherein the one or more outlets are fluidly coupled to lytic agents, and wherein the arterial space is a pulmonary bay caused by the pulmonary embolism.

In yet another aspect of the present invention, a method treating a patient with a pulmonary embolism, the method includes the following: inserting the above-mentioned device over a wire until the aspirator inlet passes through the pulmonary bay at least adjacent to the pulmonary embolism; moving the expandable balloon to the expanded condition until a seal is formed in the pulmonary bay between a segment of arterial wall and the expandable balloon; selectively urging lytic agents through the one or more outlets; and generating suction through the aspirator inlet.

These and other features, aspects and advantages of the present invention will become better understood with reference to the following drawings, description and claims.

### BRIEF DESCRIPTION OF THE DRAWINGS

FIG. 1 is a schematic view of an exemplary embodiment of the present invention;

FIG. 2 is a schematic view of an exemplary embodiment of the present invention;

FIG. 3 is a schematic view of an exemplary embodiment of the present invention; and

FIG. 4 is a flow chart of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a transcatheter treatment device for pulmonary embolism and a method of using the same. The treatment device has a conduit extending between an expandable balloon and an aspirator inlet, therebetween the conduit provides one or more outlets fluidly coupled to lytic agents. The expandable balloon is adapted to be actuated between a collapsed condition and an expanded condition dimensioned to seal off a portion of the interior of the pulmonary artery. In the collapsed condition the treatment device is percutaneously inserted into the pulmonary artery until the aspirator inlet is adjacent the pulmonary embolism, wherein the expandable balloon actuates to the expanded condition sealing off a pulmonary bay between the expanded balloon and the occluding blockage, as which time the practitioner can selectively deliver lytic agents and suction into this closed system space of the pulmonary artery in such a way as to disintegrate the clot.

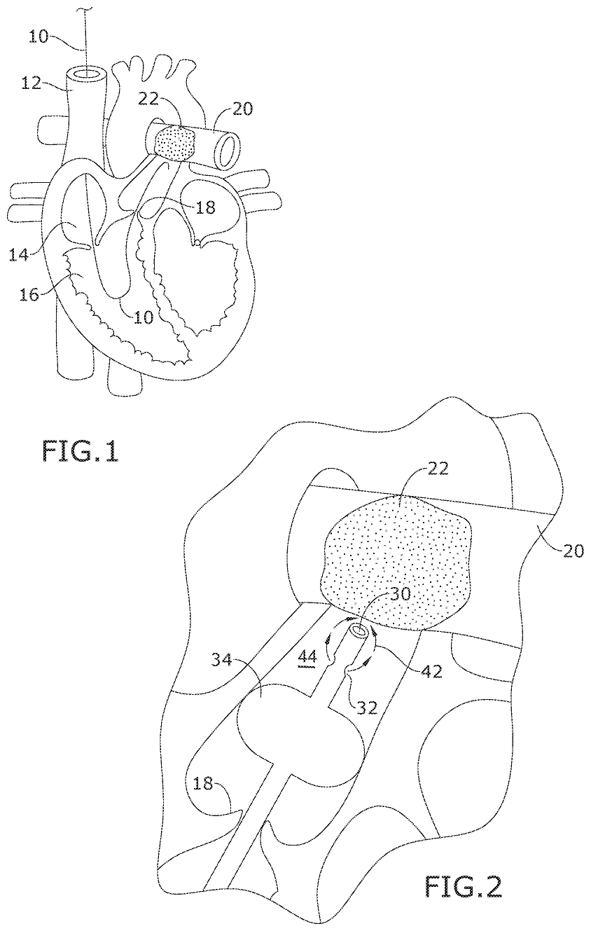

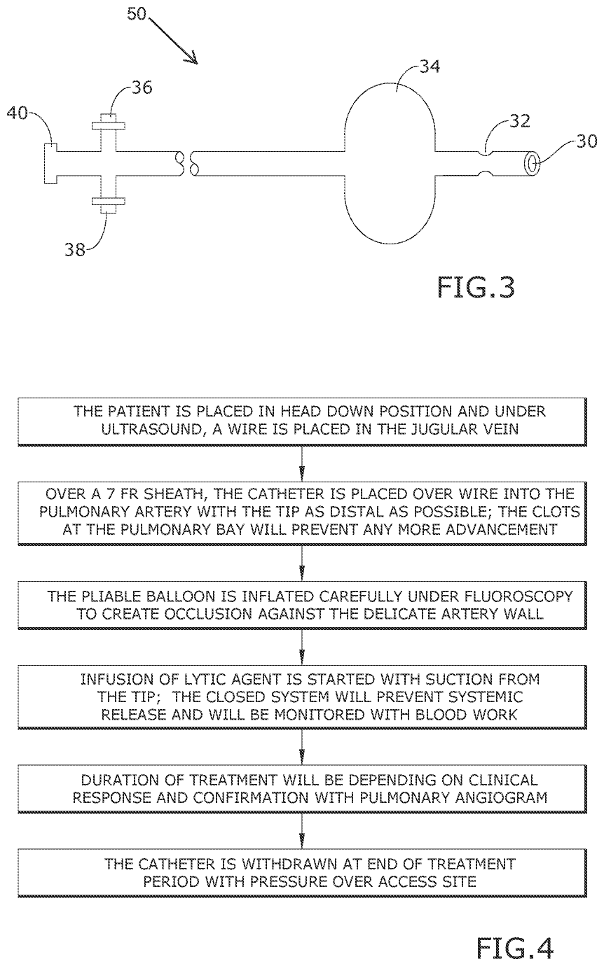

Referring now to FIGS. 1 through 4, the present invention may include an endovascular, rapid-access, balloon catheter for treatment of pulmonary embolism. The present invention embodies a single closed system of over-the-wire transcatheter balloon and infusion/suction channels.

This single system embodies the following steps: first, the patient with the pulmonary embolism 22 may be placed in the prone position. In certain embodiments, under ultrasound, fluoroscopic guidance, or equivalent a wire 10 may be urged through an upper body access point 12, via the jugular vein, through the right atrium 14 and right ventricle 16 and through the pulmonary valve 18. Over a suitably sized sheath, e.g., 7 Fr sheath, the catheter is placed over the wire 10 into the pulmonary artery 20 with the tip as distal as possible. The clots at the pulmonary bay 44 will prevent any more advancement. The pliable balloon is expanded carefully under fluoroscopy to create occlusion against the delicate wall of the pulmonary artery 20, thereby defining a closed system or space within the pulmonary bay 44. Infusion of lytic agent may be started through the side outlet(s) 32 in concert with suction from the tip outlet 30, whereby the combination urges an infusion pathway 42 that is evocative of magnetic field lines forming concentric (semi)circles around the cylindrical distal end of the systemic device 50, due to the polar relationship between the distal inlet 30 and the diametrically opposing side outlets 32. The closed system will prevent systemic release and will be monitored with blood work. Duration of treatment will depend on clinical response and confirmation with pulmonary angiogram. The catheter may be withdrawn at end of treatment period with pressure over access site.

The systemic device 50 may be made from medical grade plastics. The expandable balloon may be pliable and soft with a disc shape to allow compression of the pulmonary artery 20. The ultrasound guided wire placement will be used to introduce the balloon catheter into the pulmonary artery 20. Once in place, the balloon is carefully expanded and lytic infusion cycle started with suction at the tip/inlet 30. The systemic device 50 may have a low-pressure compliant expandable balloon 34 and an operatively associated balloon port 40 for moving the expandable balloon between a collapsed condition and the expanded condition. An infusion port 38 and a suction port 36 may be operatively associated with the and the side outlet(s) 32 and the aspirator inlet 30, respectively.

Suction may be provided at a negative pressure of between 200-600 mm Hg with 400 mm Hg preferred. As used herein, the terms “vacuum” or “suction” refer to negative pressure relative to atmospheric or environmental air pressure. Suction may be provided via one or more manual or electric pumps, syringes, suction or squeeze bulbs or other suction or vacuum producing means, devices or systems. Suction source may comprise one or more vacuum regulators, resistors, stopcocks, connectors, valves, e.g., vacuum releasing valves, filters, conduits, lines, tubes and/or hoses. The conduits, lines, tubes, or hoses may be flexible or rigid. For example, a flexible suction line may be used to communicate suction to a tissue-engaging device.

In certain embodiments, the catheter is a unibody device with three separate channels and access ports. The main channel is the largest and is used for the over wire to introduce the device in the target within the vein and to suction clots out. The side port is 2-3 cm and proximally placed which allows for the introduction of chemical lytic agents. The third port allows the inflation of the balloon 2-3 cm proximally from side port. The balloon can be expanded as needed and allows the tip to press against clots and keeping the lytic agents mostly concentrated within the desired zone.

The catheter may be introduced over a wire into the internal jugular vein until it reaches the obstruction due to pulmonary embolus whereat the balloon is inflated to keep the tip aligned against the clots. The suction may be started with aspiration of clots after the wire is removed. The lytic agents may be infused as infusion which can be few boluses at the beginning and most of this will be suctioned back limiting systemic effects. An arterial line and a lower extremity central line are also used to detect higher levels in arterial blood indicating dissolution of clots. A peripheral vein catheter is used for systemic heparinization.

This unibody device would be made from medical grade non-thrombogenic plastic which should be of standards typically applied to endovascular devices used commercially available.

A method of using the present invention may include the following. A medical practitioner, at bedside, may place the ultrasound guided (0.035″) floppy end wire through a micro-puncture of the internal jugular vein into the pulmonary artery near the clot. The catheter may be introduced under echocardiogram and fluoroscopy guidance with the tip placed as close to the target clot as possible. The balloon is gently inflated to allow antegrade blood flow to push the catheter tip against the clot. The wire may be removed, and intermittent suction started. The side port is used to start infusion of lytic agents starting with boluses and transitioning to slow infusion later depending on patient response. Contrast agents or carbon dioxide can be injected intermittently from tip of catheter to get real time information of clot burden and location, in between aspiration. The location of clot can be used to move the tip further in to pulmonary artery with over the wire guided by fluoroscopy. The arterial line in the upper extremity may be used to check systemic lytic agent levels, thereby monitoring the response to treatment while systemic heparin is administrated over a peripheral vein. Real time electrocardiography and echo allows assessment of cardiac function. The treatment duration may be short with extraction of most of the thrombus or prolonged over few hours in the need for slow sustained response, particularly in a sick patient.

It should be understood, of course, that the foregoing relates to exemplary embodiments of the invention and that modifications may be made without departing from the spirit and scope of the invention as set forth in the following claims.

What is claimed is:

1. A device for endovascular treatment for a patient with a pulmonary embolism, the device comprising:
a conduit extending between an expandable balloon and an aspirator inlet;
the expandable balloon selectively movable between a collapsed condition and an expanded condition dimensioned to seal an arterial space defined by the pulmonary artery of the patient; and
one or more outlets circumferentially disposed along the conduit between the expandable balloon and the aspirator inlet.

2. The device of claim 1, wherein the aspirator inlet generates suction.

3. The device of claim 2, wherein the one or more outlets are fluidly coupled to lytic agents.

4. The device of claim 3, wherein the arterial space is a pulmonary bay caused by the pulmonary embolism.

5. A method treating a patient with a pulmonary embolism, the method comprising:
inserting the device of claim 4 over a wire until the aspirator inlet passes through the pulmonary bay at least adjacent to the pulmonary embolism;
moving the expandable balloon to the expanded condition until a seal is formed in the pulmonary bay between a segment of arterial wall and the expandable balloon;
selectively urging lytic agents through the one or more outlets; and
generating suction through the aspirator inlet.

6. The method of claim 5, wherein the urged lytic agents travel an orbital pathway from the one or more outlets to the aspirator inlet.
